(19) United States
(12) Patent Application Publication
(10) Pub. No.: US 2011/0125136 A1
(43) Pub. Date: May 26, 2011

(54) INTRAVASCULAR DIAGNOSTIC AND THERAPEUTIC SAMPLING DEVICE

(76) Inventors: Morteza Gharib, San Marino, CA (US); Derek Rinderknecht, Arcadia, CA (US); Danny Petrasek, Los Angeles, CA (US)

(21) Appl. No.: 13/013,733

(22) Filed: Jan. 25, 2011

Related U.S. Application Data
(62) Division of application No. 11/450,862, filed on Jun. 8, 2006.
(60) Provisional application No. 60/688,436, filed on Jun. 8, 2005.

(51) Int. Cl. A61M 5/00; C12Q 1/02; C12Q 1/54; C12Q 1/60

(52) U.S. Cl. 604/890.1; 435/29; 435/14; 435/11

(57) ABSTRACT

A device and methods for continuous precision blood sampling from a patient with a micro impedance pump as the driver in a microfluidic system. Depending on the needs of medical technologies, the micro impedance pump in the intravascular diagnostic and therapeutic sampling system serves in a forward pumping function for blood sampling, in a backward infusing for therapeutic treatment, and in a valving function for controlling fluid flow.

### CROSS-REFERENCE TO RELATED APPLICATIONS

This application claims priority benefits of provisional application Ser. No. 60/688,436, filed Jun. 8, 2005, entitled “Intravascular Diagnostic and Therapeutic Sampling Device”, the entire contents of the provisional application are incorporated herein by reference.

### FIELD OF THE INVENTION

The present invention is generally related to an in vitro blood sampling system, more particularly, the invention relates to an intravascular blood sampling device with continuous precision sampling for medical technologies.

### BACKGROUND OF THE INVENTION

In many medical settings and specifically during hospitalizations, there is often a need to monitor the levels of many important physiological indices in the blood. For example, the levels of all the major electrolytes (Na, K, Cl, HCO, Ca, Mg, and the like) as well as the level of glucose and other measured metabolic and physiological markers routinely used by physicians to make diagnosis and guide therapy. In the intensive care unit (ICU) there is even more urgency to closely monitor these levels and as such the manpower and resources to perform these blood sampling tests are expensive and time consuming. The costs of testing and monitoring are compounded today by all of the other expensive facets in health care delivery and the crisis is only worsening. Additionally, in the case of extended ICU care, there is a great deal of blood that is sampled for diagnostics that could actually begin to exacerbate anaemic status.

U.S. Pat. No. 6,254,355 to Gharib, the entire contents of which are incorporated herein by reference, discloses a valveless fluid system based on pinch-off actuation of an elastic tube channel at a location situated asymmetrically with respect to its two ends. Means of pinch-off actuation can be either electromagnetic, piezoelectric, pneumatic, mechanical, or the like. A critical condition for the operation of the “hydro-elastic pump” therein is in having the elastic tube attached to other segments that have a different compliance (such as elasticity). This difference in the elastic properties facilitates elastic wave reflection in terms of local or global dynamic change of the tube's cross-section that results in the establishment of a pressure difference across the actuator and thus a net unidirectional movement of fluid. The intensity and direction of this flow depends on the frequency, duty cycle, and mechanical properties of the tube.

U.S. Pat. No. 6,585,660 to Dorando et al., entire contents of which are incorporated herein by reference, discloses a signal conditioning device having low power requirements and a simplified connection scheme for interfacing intravascular diagnostic devices, such as a pressure sensor disposed upon a distal end of a guide wire, and a physiology monitor providing an excitation signal for the intravascular diagnostic devices. Furthermore, a system for taking a measurement from within a blood vessel to determine a flow characteristic within the blood vessel, the system comprising a flexible elongate member having a sensor mounted thereon; a cable electrically connecting the sensor to a signal conditioning device; a processing unit for performing programmed tasks; a physiology monitor interface including an input for receiving an excitation signal from the physiology monitor, and an output for transmitting an output measurement signal to the physiology monitor generated in accordance with the sensor measurement signal; and a power supply circuit including a signal converter that energizes at least the processing unit with power supplied by the excitation signal.

U.S. Pat. No. 6,679,687 to Gharib, entire contents of which are incorporated herein by reference, discloses a pump comprising: first and second elastic chambers, the first elastic chamber having a first fluidic characteristic which is different than a second fluidic characteristic of the second elastic chamber; and a pressure increasing element, which induces a pressure increase into the first and second elastic chambers that causes a pressure difference between the first and second chambers that is based on the different fluidic characteristic differences of the chambers, to cause a pumping action based on the pressure difference.

U.S. Pat. No. 7,011,508 to Lum, entire contents of which are incorporated herein by reference, discloses an apparatus and method for making a microscopic paddle wheel coupled inductively by an external electromagnet and used for valving and active pumping so that the actual pumping mechanism is completely isolated from the electromagnetic driver. A cartridge having a network of conduits and reservoirs contains several of such paddle wheels to transport blood and reagents. A point-of-care device houses the electromagnetic driving mechanism and is reused with successive cartridges since the paddle wheels are contained by the cartridge and do not contaminate the driving mechanism.

U.S. Patent Application Publication No. 2005/0015001 to Lec et al., entire contents of which are incorporated herein by reference, discloses an acoustic blood analyzer comprising a blood sampling means to obtain a blood sample, a fluidic section to deliver and distribute a blood sample to the acoustic sensor; an electronic section means which excites the sensor and detects changes in the operational parameters of the transducer section; and a packaging section which provides mechanical and functional integrity to the transducer, fluidic and electronic section means of the analyzer as well as an interface for the analyzer with analytical laboratory systems and computer based data processing, storage and display systems.

Joo and associates (NSTI Nanotechnology Conference, May 8-12, Anaheim, Calif., pg. 213-214) reported a microfluidic system with non-enzymatic glucose sensor based on nano-porous platinum and platinum/platinum oxide reference electrode, which is integrated with a microfluidic chip. The microfluidic chip is comprised of microfluidic transport channels, reservoirs, an electrochemical reaction chamber, and pumping means by applying high voltage to the electrode under the corresponding reservoir.

Zahn and associates reported an integrated microfluidic device for the continuous sampling and analysis of biological fluids (Proceedings of 2001 Microfluidic ASME International Mechanical Engineering Congress and Exposition, Nov. 11-16, 2001, New York, pg. 3-6). The integrated microfluidic system includes the assembly of microdialysis microneedles (capable of excluding large molecular weight protein compounds) with on-chip flow channels and electronics with positive displacement micropumps, microvalves and a planar electrochemical sensor for biological detection. The planar microfluidic system is capable of sampling and analyzing biological solutions, sensor cleaning and recalibration.

U.S. Pat. No. 7,025,323 to Krulevitch et al., entire contents of which are incorporated herein by reference, discloses a microfluidic system having at least one microchannel, the microchannel comprising a plug-actuation device and a pressure generation mechanism adapted to drive the plug of the plug-actuation device to slide along the microchannel for pumping and for valving.

U.S. Pat. No. 6,989,128 to Alajoki et al., entire contents of which are incorporated herein by reference, discloses an apparatus for modulating flow rates in microfluidic devices by modulating downstream pressure in the device to change the flow rate of materials in an upstream region of the device. Such methods include electrokinetic injection or withdrawal of materials through a side channel and the use of an absorbent material to induce wicking in the channel system.

What is clinically needed is a simple microfluidic device that could be used to precisely sample blood volume and continuously monitor vital blood parameters so that it would alleviate the need for manual blood sampling and enable a significant reduction the volume of samples taken.

### SUMMARY OF THE INVENTION

It is one object of the present invention to provide a method and a system for the continuous precision sampling of pico/nano liter blood samples for medical technologies such as metabolite and other physiological indicators. This system is comprised of a sample well array, fluidically connected valves, a cleansing reagent reservoir, one or more micro impedance pumps as well a waste reservoir. The novelty of the system is that the device is configured to work with existing intravenous catheters or other systems granting intravenous access, reducing the manpower and time committed to taking diagnostic samples such as blood glucose and the inclusion of the micro impedance pump as a driver for the system.

Some aspects of the invention provide a microfluidic system having at least one microchannel and a grid of sample wells, the improvement comprises a micro impedance pump in at least one microchannel, the micro impedance pump having a first fluid driven capability for providing precise fluid quantity to the grid of sample wells for diagnosis, wherein the micro impedance pump device also has a fluid valving capability for flow control.

Some aspects of the invention provide a method of continuously or intermittently monitoring physiological indices or markers of a patient, comprising: a) providing a micro fluidic system having at least one microchannel, a fluid reservoir for flushing, a grid of sample wells, and a first micro impedance pump in at least one microchannel; b) initiating the first micro impedance pump for a predetermined duration or by the use of a flow sensor in a manner to supply a precise amount of blood through the at least one microchannel to at least one sample well for sample analysis; c) flushing the at least one microchannel and the sample wells to rid of waste fluid; and d) repeating steps b and c to continuously or intermittently monitor the physiological indices or markers of the patient.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawings.

FIG. 1 shows one aspect of the continuous blood sampling device with a micro impedance pump as a driver or valve for the system.

FIG. 2 shows one simulated aspect of the micro impedance pump system with a pinching element in operation.

FIG. 3 shows another simulated aspect of the micro impedance pump system with a magnet-pinching element in operation.

FIG. 4 shows a schematic diagram of the intravascular diagnostic and therapeutic device of the present invention for diagnosis and therapy functions.

### DETAILED DESCRIPTION OF THE EXEMPLARY EMBODIMENTS

The preferred embodiments of the present invention described below relate particularly to an intravascular diagnostic and therapeutic sampling device for a patient. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

Micro Impedance Pump

In one embodiment, a micro impedance pump is operated under the principles of utilizing an elastic tube element with attached end members having different hydroimpedance properties, wherein the elastic element is pinched with certain frequency and duty cycle to form asymmetric forces that pump fluid in a desired direction. Unlike peristaltic pumps, this impedance pump does not necessarily implement complete squeezing or forward displacing by a squeezing action. Additionally, the impedance pump does not respond linearly to increasing actuation frequency as in the peristaltic case.

In a co-pending application, U.S. Patent Application Publication No. 2004/0101414, it is disclosed a valveless pump comprising an elastic element having a length with a first end and a second end, and a first end member attached to the first end of the elastic element and a second end member attached to the second end, wherein the first end member has an impedance different from an impedance of the second end member. In one preferred embodiment, the pump further comprises pressure change means for inducing a pressure increase and a pressure decrease into the first and second end members, in a way that causes a pressure difference between the first and second end members, and causes a pumping action based on the pressure difference.

In a co-pending application, U.S. Patent Application Publication No. 2005/0275494, it is disclosed an electromagnetic actuator for a micro fluidic pump of the type that causes periodic pinching and releasing against the walls of a fluidic channel, e.g., a tube. At least one permanent magnet is placed against the walls of the fluidic channel, and located in an area with magnetic fields, produced by coils that are radially symmetric to the channel. The permanent magnet is caused to press and release against the wall of the fluid channel to cause a fluid flow through the channel.

In a co-pending application, U.S. Patent Application Publication No. 2005/0277865, it is disclosed a medical application using the micro impedance pump as the driver for fluid transport. One aspect of the co-pending application discloses a method for treating hydrocephalus comprising: using a tubular shaped catheter shunt system with a collector portion, a discharge portion, and a pump portion, all of which are connected to one another, and all of which are substantially tubular in shape; implanting the system into a human patient, with a tip section disposed in brain ventricles of the patient, and an end section disposed in a body cavity of the patient; and actuating the tubular shaped hydroimpedance pump to pump fluid from the brain ventricles into the body cavity.

The elastic wave reflection of a hydro-elastic pump depends on the hydro impedance of the segments on either side of the pump. In the prior art hydro-elastic pump, for example, U.S. Pat. No. 6,254,355, it was required that the segments to be stiffer either by using a different material or using reinforcement. To overcome the limiting conditions of the prior hydro-elastic pump systems, it is disclosed herein that the pinching location separates two segments with different hydroimpedances, including but not restricted to the characteristic impedance or any impedance in which attenuation occurs over distance, with certain frequency and duty cycle to form asymmetric forces that pump fluid achieving a non-rotary bladeless and valveless pumping operation.

Periodic excitation of the micro impedance pump allows a small volume of blood to be taken and sequestered that allows continuous monitoring of various physiological indices within the blood. The microimpedance pump can be operated without the need for bulky high-pressure pneumatic drivers. By completely closing the chamber section of the impedance pump, the pump itself can function as a valve. This system could also be implemented within a feedback loop to be used in combination with various glucose metering and therapeutic dosing strategies, including insulin.

Some aspects of the invention relate to a tubular hydroimpedance pump that is useful and applicable in medical and biomedical applications, such as intravenous diagnostic and therapeutic sampling microfluidic system and any other application that requires controlled pumping for medical purposes.

Microfluidic System

In one embodiment, the micro impedance pump could be placed in either an intravenous or intra-arterial catheter or function as the catheter itself. In another embodiment, the micro impedance pump could lie outside the vein or artery and be fluidically coupled to the catheter. In one preferred embodiment, the micro impedance pump is modular. The function of this pump would be to draw small volumes of blood at specified intervals and deliver the sample to an attached diagnostic grid. This diagnostic sample grid may be integrated into a microfluidic system containing the micro pump as a single device, or be housed within a replaceable cartridge that connects to the micro impedance pump for sample delivery. In a further embodiment, the device may also be used in similar configurations with arterial catheters.

In operations, a heparin or anticoagulant reservoir could also be integrated into the device to take advantage of the reversibility of the pump allowing for frequent redirection and purging of any contaminants as well as to resist clotting. Additional components such as thermoelectric coolers can also be integrated to preserve and store the samples for future use. The following example is only illustrative of an application.

Hospitalized patients with poorly controlled diabetes mellitus (DM) must have their glucose level sampled very frequently so that the dosage of therapy (insulin) can be more accurately adjusted. In fact, without the frequent monitoring of the glucose, the glucose level can fall below the dangerous level without signs of symptom. Currently monitoring the glucose is done manually by nurses and due to shortage in personnel, it is difficult to sample blood more frequently than in 1-2 hours intervals. In particular, in intensive care and other specialized settings the demand for a nurse's time is strained. Currently, phlebotomists and nurses are occupied with the task of drawing and measuring glucose levels and as such the time devoted to other nursing needs is significantly impacted. The intravenous diagnostic and therapy sampling device of the present invention can be employed in an ideal way to address this issue. Moreover, the device disclosed would benefit the patients directly by avoiding the need to wake up a patient in order to perform the measuring task.

Blood Sampling Device

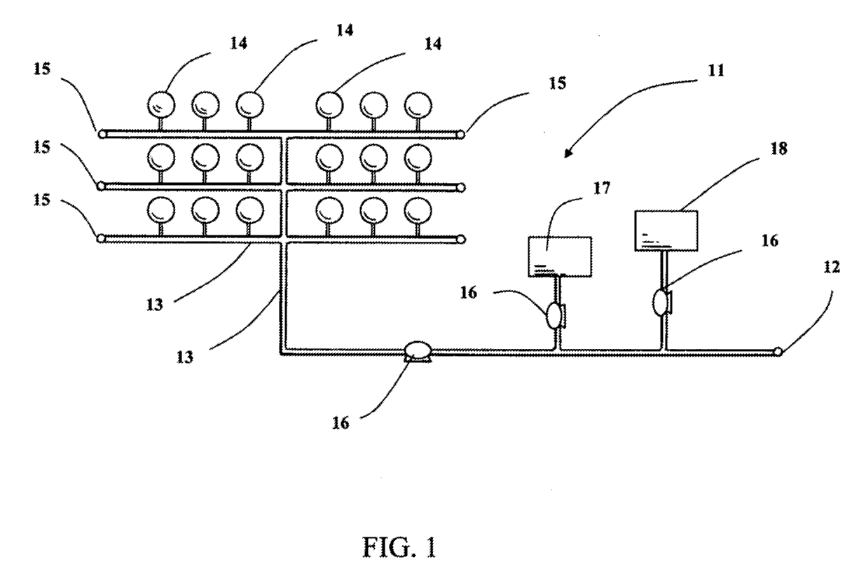

FIG. 1 shows an arrangement for the continuous blood sampling device 11 with a micro impedance pump 16 as a driver or valve for the system. A small pump/catheter system made of suitable biocompatible material and dimensions (for example, Parylene) is inserted in a peripheral vein at an inlet end 12 for intravascular blood access. A chamber of heparin (anticoagulant) 17 and/or heparinized saline 18 can be used to intermittently flush the chamber, sampling grid, or the microchannels 13 and to keep the blood from clotting. The inlet fluid from chamber 17 or 18 can be precisely controlled via a micro impedance pump 16 for pumping and valving. The waste is flushed through the waste purging line 15. A replaceable grid of small wells 14 could be used for storing the blood and for glucose sampling. Such a device would be able to monitor glucose levels at specified time intervals without the need of the nurse's assistance. The valves sealing the sample wells are not shown. In one embodiment, the device can be connected to a computer with display readouts for glucose results automatically and instantly. The device as shown in FIG. 1 could be used to also deliver insulin or other therapeutic drugs through the vein as a drip or in pulses by using the micro impedance pump 16 as the control in an opposite flow direction toward the inlet end 12.

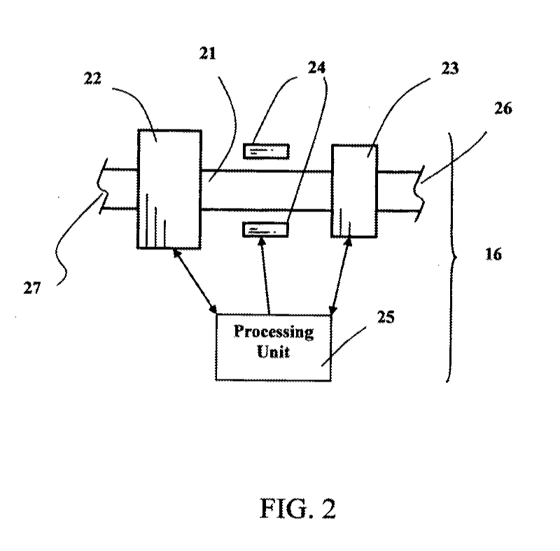

An impedance pump 16 with a processing unit 25 is shown in FIG. 2 to illustrate one aspect of the principles of continuous precision sampling of pico/nano blood samples for diagnostics. In one embodiment, the flow pump system 16 comprises a feedback control processing unit 25 to initiate and regulate the blood flow in the fluid channel from a first end 26 to a second end 27 through the pump element. The pump system 16 comprises an elastic tube element 21 having two end members 22, 23, wherein the elastic properties of the elastic tube element 21 are substantially uniform along the full length between the end members. The elastic tube element 21 has an impedance Z₁ whereas the end members 22 and 23 have impedances Z₂ and Z₃, respectively. In general, Z₁ is different from either Z₂ or Z₃. The impedance, Z is a frequency dependent resistance applied to a hydrofluidic pumping system defining the fluid characteristics and the elastic energy storage of that segment of the pumping system. By varying the excitation profile parameters for example, the duty cycle, frequency, waveform and offset the flow rates and flow directions can be manipulated and precisely controlled.

As shown in FIG. 2 and previously described in a co-pending application, U.S. Patent Application Publication No. 2005/0277865, the fluid in the elastic tube unit moves from the first end 23 to the second end 22 by applying force to the pinching element 24. The pinching element 24 is driven by a programmable driver or other means which is incorporated in or attached to the processing unit 25, wherein the unit 25 displays the flow/pressure data and at least one of frequency, phase and amplitude of the driving. The feedback system may include a flow and pressure sensor on the elastic tube element 21 (not shown). The values as provided to control the timing, frequency and/or amplitude of the pinching via feedback. The relationship between timing, frequency, and displacement volume for the compression cycle can be used to deliver the required performance. The intensity and direction of this flow depends on the frequency, duty cycle, and elastic properties of the tube unit. The system also can include a flow control system. IN one embodiment of such a system, the impedance pump can be used as a valve to control fluid flow, and a sensor can be used to determine the amount of fluid delivered. Thus, fluids can be precisely delivered through both control of time at a determined delivery rate, or by controlling the flow and measuring with a sensor.

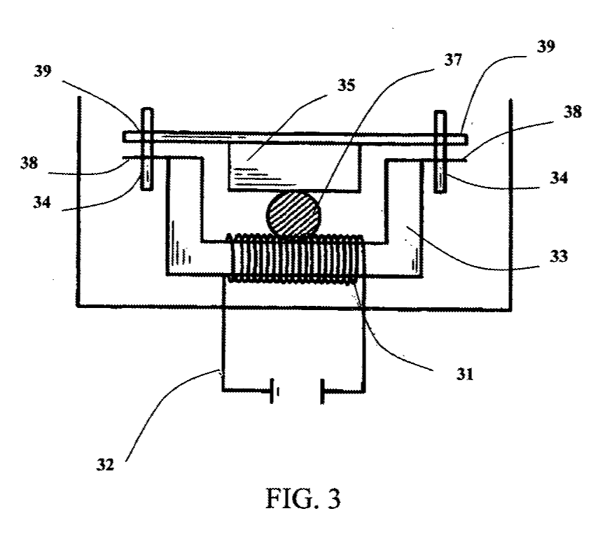

FIG. 3 shows an impedance pump system where a magnet 31 has a substantially U-shaped yoke 33 that provides a magnetic force that pulls the pincher element 35 on bearings 34. This system can be advantageous, for many reasons. The bearings 34 can be formed in a simple and reliable way, since they only require back and forth motion. They can be spring-biased. Alternatively, they can operate without spring bias. In addition, if the plunger element 35 is non-magnetic, then the magnetic force is between the ends 38 of yoke 33 and its attractive element 39. When this happens, no magnetic force is provided through the tube 37.

A number of different alternatives for pressure pinching are also contemplated as described in co-pending applications, such as U.S. application publication no. 2003/0233143, no. 2004/0101414, no. 2005/0275494, and no. 2005/0277865. In addition, a number of improvements are expected. This system can be used for pumping blood for blood sampling. In contrast with existing blood flow systems, such as those used in traditional left ventricle devices, this system does not require any valve at all, and certainly not the complicated one-way valve systems which are necessary in existing devices. This can provide a more reliable device, since any mechanical constrictions in the blood stream provide a potential site for mechanical failure as well as sedimentation of blood and thrombosis. Hence, this system, which does not require a valve system, can be highly advantageous.

In addition, the compression frequencies of this system can operate below 5 cycles per second for a microfluidic blood sampling or drug therapeutic system. This has an advantage over modern blood pumps that may require up to 90,000 rotations per minute/1,500 cycles per second of up to 16 blades to propel the blood.

Some aspects of the present invention relate to a microfluidic system having at least one microchannel, a grid of sample wells, at least one micro impedance pump device in the at least one microchannel, the micro impedance pump device having a first fluid driven capability for providing precise fluid quantity to the grid of sample wells for diagnosis, wherein the micro impedance pump device also has a fluid valving capability for providing the precise fluid quantity or no flow as a stopper. In a further aspect, the micro impedance pump device comprises a second fluid driven capability that drives fluid in an opposite direction as of the first fluid driven capability, for infusing therapeutic fluid to the patient, wherein a distal end of the at least one microchannel is configured accessible to a blood vessel of a patient. The microfluidic system of the invention may further comprise an anticoagulant reservoir for releasing anticoagulant to blood sample received from the patient, wherein the releasing of anticoagulant is provided by a second micro impedance pump.

Within the past decade significant progress has also been made in the field of microelectromechanical systems (MEMS). MEMS is a class of systems that are physically very, very small. These systems typically, but not exclusively, have both electrical and mechanical or optical components. MEMS devices could be incorporated in a micro fluidic system of the present invention for a variety of sensing and actuating functions with intravascular blood access from a patient. MEMS devices have been conceived for typing blood, counting cells, identifying DNA, performing chemical assays, measuring pH, sensing partial pressures, and performing a wide variety of other procedures and tests. Recently, a “lab on a chip” has been developed that is suitable for carrying out a variety of blood or blood constituent assays or tests relating to the microfluidic blood sampling system with the micro impedance pump as a driver or valve for the system.

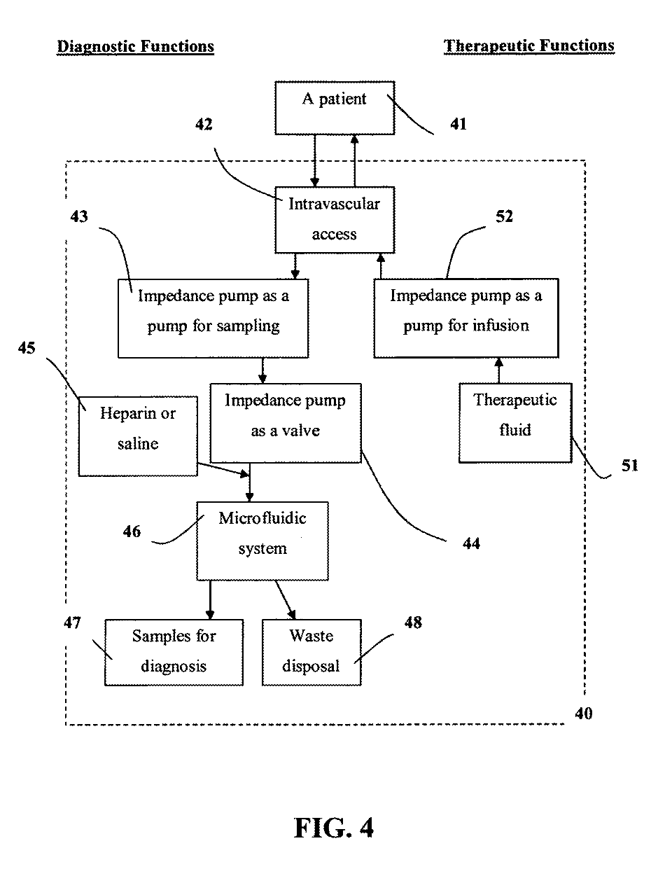

FIG. 4 shows a schematic diagram of the intravascular diagnostic and therapeutic device 40 of the present invention for diagnosis and therapy functions. In operations, an intravascular access 42 is established between the device and a patient 41. A micro impedance pump 43 as a pump for blood sampling is installed in the fluid channel. The same pump 43 or a new impedance pump 44 may serve as a valve so to control the blood sampling quantity and to prevent fluid from back-flowing to the patient 41 when a therapeutic procedure is needed. A conventional microfluidic system 46 with heparin/saline reservoir 45 options for diagnosis 47 and waste disposal 48 is part of the overall device system 40. For therapeutic procedures, therapeutic fluid 51, such as insulin, chemotherapy fluid, painkiller, or antibiotic etc., can be infused into the patient 41 using the existing pump at a reversed flow direction or a new impedance pump 52 for precise and continuous or intermittent fluid therapy.

In summary, some aspects of the invention relate to a method of continuously or intermittently monitoring physiological index or marker of a patient, comprising at least some of the following steps of (a) providing a microfluidic system having at least one microchannel, a fluid reservoir for flushing, a grid of sample wells, and a first micro impedance pump in the at least one microchannel; (b) initiating the first micro impedance pump for a predetermined duration or through the use of a flow sensor in a manner to supply a precise amount of blood through the at least one microchannel to at least one sample well for sample analysis; (c) flushing the at least one microchannel and the sample wells to rid of waste fluid; and (d) repeating steps b and c to continuously or intermittently monitoring the physiological index or marker of the patient. In one preferred embodiment, the physiological index is a level of electrolytes selected from a group consisting of Na, K, CI, HCO, Ca, Mg, and the like, and combinations thereof. In another embodiment, the physiological marker is a level of glucose, cholesterol, C-reactive protein, and the like.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

1.-13. (canceled)

14. A method of continuously or intermittently monitoring a physiological index or marker of a patient, comprising:
(a) providing a microfluidic system having at least one microchannel, a fluid reservoir for flushing, a grid of sample wells, and a micro impedance pump in said at least one micro channel;
(b) initiating the micro impedance pump for a predetermined duration or by a flow control sensor in a manner to supply a precise amount of blood through the at least one microchannel to at least one sample well for sample analysis;
(c) flushing the at least one micro channel and the sample wells to rid of waste fluid;
(d) repeating steps b and c; and
(e) analyzing the blood in the at least one sample well for the physiological index or marker.

15. The method of claim 14, wherein the physiological index is a level of electrolytes selected from a group consisting of Na, K, Cl, HCO, Ca, Mg, and combinations thereof.

16. The method of claim 14, wherein the physiological marker is selected from the group consisting of glucose, cholesterol, and C-reactive protein.

17. The method of claim 14, wherein the micro impedance pump comprises an actuator to drive said pump.

18. The micro fluidic system of claim 17, wherein the actuator is selected from the group consisting of a piezoelectric actuator, an electromagnetic actuator, a pneumatic actuator, a polymeric actuator, and a hydraulic actuator.

19. The method of claim 14, wherein the micro impedance pump serves in a forward pumping function for blood sampling, in a backward infusing function for therapeutic treatment, and in a valving function for controlling fluid flow.
